(19) United States
(12) Patent Application Publication
(10) Pub. No.: US 2020/0385347 A1
(43) Pub. Date: Dec. 10, 2020

(54) PYRROLE COMPOUND

(71) Applicant: MYCOLOGY TECHNO.CORP., Niigata (JP)

(72) Inventors: Seiichi MATSUGOU, Ishikawa (JP); Toshio SAKAMOTO, Ishikawa (JP); Ayaka NISHIDA, Ishikawa (JP); Naoki WADA, Ishikawa (JP); Tetsuya KONISHI, Niigata (JP)

(21) Appl. No.: 16/768,425

(22) PCT Filed: Nov. 26, 2018
(86) PCT No.: PCT/JP2018/043401

§ 371 Date: May 29, 2020

(30) Foreign Application Priority Data
Nov. 29, 2017 (JP) 2017-229598

(51) Int. Cl. C07D 207/337

(52) U.S. Cl. CPC C07D 207/337

(57) ABSTRACT

The present invention provides a pyrrole compound represented by the following formula (1):

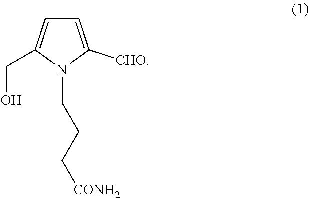 [SMILES: NC(=O)CCCn1c(C=O)ccc1CO]

### TECHNICAL FIELD

The present invention relates to a novel pyrrole compound. More particularly, the invention relates to a pyrrole compound, which is a novel substance contained in an extract composition of Basidiomycetes-X FERM BP-10011.

### BACKGROUND ART

Since old days, mushrooms have been frequently used as food materials having unique flavors and tastes. Having physiological function activating actions, such as enhancement of immunocompetence, antimicrobial activity, control of biorhythm, and prevention of senescence, mushrooms have also been used as Chinese herbal medicines or folk medicines for certain types of diseases. Studies of pharmacological ingredients concerned with mushrooms are in progress, resulting in the discovery of ingredients exerting antibacterial and antiviral actions, a cardiotonic action, a hypoglycemic action, a cholesterol-lowering action, an anti-thrombotic action, and an anti-hypertensive action.

The present applicant previously found a novel fungus Basidiomycetes-X FERM BP-10011 (hereinafter referred to simply as “Basidiomycetes-X”) and filed a patent application on an extract composition thereof (hereinafter referred to as a “Basidiomycetes-X extract composition”) (see Patent Document 1). The Basidiomycetes-X extract composition, containing a large amount of polysaccharide (β-D-glucan), exhibits high anti-oxidative power and OH radical-scavenging action. Thus, the composition is expected to exhibit an anti-aging action and the like. The Basidiomycetes-X extract composition, having an immunomodulating action, is suitably used as an immunoactivator or the like. Also, the present applicant previously filed a patent application on a composition for ameliorating/preventing an atopic disease, which composition is based on the Basidiomycetes-X extract composition (see Patent Document 2).

### PRIOR ART DOCUMENTS

### Patent Documents

Patent Document 1: WO 2004/097007

Patent Document 2: Japanese Patent Application Laid-Open (kokai) No. 2007-109449

### SUMMARY OF THE INVENTION

### Problems to be Solved by the Invention

Patent Documents 1 and 2 suggest that a Basidiomycetes-X extract composition may contain a novel substance. However, such a candidate compound has not been identified in the documents. If a novel substance contained in the Basidiomycetes-X extract composition is identified, use of the novel substance would be encouraging.

Under such circumstances, an object of the present invention is to provide a novel substance contained in a Basidiomycetes-X extract composition.

### Means for Solving the Problem

The present inventors have conducted extensive studies in order to attain the above object, and have found that a Basidiomycetes-X extract composition contains a novel substance. The novel substance has been isolated and purified, and the structure thereof has been characterized. The present invention has been accomplished on the basis of this finding.

A first mode of the present invention so as to attain the aforementioned object provides a pyrrole compound represented by the following formula (1).

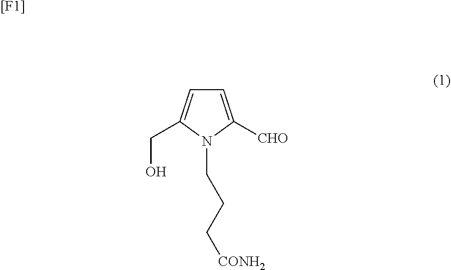

### Effects of the Invention

The present invention can provide a novel substance contained in a Basidiomycetes-X extract composition.

### BRIEF DESCRIPTION OF THE DRAWINGS

FIG. 1 A graph showing the total phenol amount in each extraction fraction.

FIG. 2 A graph showing the DPPH radical scavenging activity of each extraction fraction.

FIG. 3 A graph showing the iron ion-reducing performance of each extraction fraction.

FIG. 4 A graph showing the copper ion-reducing performance of each extraction fraction.

FIG. 5 A graph showing the iron ion-chelating activity of each extraction fraction.

FIG. 6 An HPLC chromatogram of an organic layer (detection wavelength: 260 nm).

FIG. 7 An HPLC chromatogram of an organic layer (detection wavelength: 290 nm).

FIG. 8 A PTLC chromatogram of an organic layer.

FIG. 9 An HPLC chromatogram of fraction 1.

FIG. 10 An HPLC chromatogram of fraction 2.

FIG. 11 An HPLC chromatogram of fraction 3.

FIG. 12 An HPLC chromatogram of fraction 4.

FIG. 13 An HPLC chromatogram of fraction 5.

FIG. 14 A DART-MS spectrum of fraction 3.

FIG. 15 A ¹H-NMR spectrum of fraction 3.

FIG. 16 A ¹³C-NMR spectrum of fraction 3.

FIG. 17 A preparative HPLC chromatogram of fraction 1.

FIG. 18 An HPLC chromatogram of fraction 1-3 and a UV-vis absorption spectrum of peak 12.

FIG. 19 A DART-MS spectrum of fraction 1-3.

FIG. 20 A ¹H-NMR spectrum of fraction 1-3.

FIG. 21 A ¹³C-NMR spectrum of fraction 1-3.

### MODES FOR CARRYING OUT THE INVENTION

The present invention is directed to a novel substance contained in an extract composition of novel fungus Basidiomycetes-X FERM BP-10011 (hereinafter referred to simply as “Basidiomycetes-X”). Hereinafter, the composition is referred to as a “Basidiomycetes-X extract composition.” The specific structure of the novel substance is represented by the following formula (1). In other words, the novel substance of the present invention is a pyrrole compound having a specific structure.

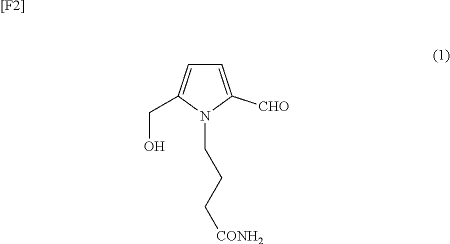

As used herein, the term “Basidiomycetes-X” refers to a basidiomycete characterized by having beaklike protrusions (i.e., clamps) but no basidium-formability, differing from other basidiomycetes. That is, even when the basidiomycete of the present invention is cultured, only sclerotia (hypha masses) are formed, but the basidia are not formed. Such a basidiomycete was obtained through retrieving a fungus from the natural world. The basidiomycete is isolated and deposited as “Basidiomycetes-X” to the NITE International Patent Organism Depositary (NITE-IPOD) of the National Institute of Technology and Evaluation (NITE) (Accession Number: FERM BP-10011).

The Basidiomycetes-X forms no conidia, or has no asexual generation. For example, when the Basidiomycetes-X is cultured in a potato glucose agar medium, the hyphae (or mycelia) formed through culturing are smooth and have clamps, but form no conidium or fruit body. Through observation of the morphology and color tone of the colony surface, light pinkish hypha masses are formed. In the case where a plurality of hypha masses are formed in a colony concentrically grown from the inoculation site, the hypha masses are interconnected via mycelial strands. Notably, the backside of the colony assumes light pink. When the Basidiomycetes-X is cultured in a glucose-dry yeast agar medium, the hyphae formed through culturing are smooth and have clamps, but form no conidium or fruit body. Through observation of the morphology and color tone of the colony surface, “light pink to white” hypha masses are formed. Hypha masses having a thickness of 5 mm to 6 mm are formed to surround the inoculation site. Notably, the backside of the colony assumes “light pink to white.”

The optimum growth conditions for Basidiomycetes-X include, for example, a pH of 5.0 to 6.0 and a growth temperature of 22° C. to 26° C. The growth allowable conditions include, for example, a pH of 4.0 to 7.5 and a growth temperature of 5° C. to 30° C.

No particular limitation is imposed on the method of culturing the Basidiomycetes-X, and the aforementioned customary method may be employed. In one exemplary mode of culturing, cultured Basidiomycetes-X cells or seed Basidiomycetes-X cells are aseptically inoculated to an agar medium, a sawdust medium, a liquid medium, or the like to which appropriate nutrient sources have been added and which has been sterilized. Culturing is performed at a suitable temperature, whereby hypha masses of the Basidiomycetes-X (hereinafter referred to as Basidiomycetes-X hypha masses) can be yielded. Notably, when cultured, the Basidiomycetes-X forms various hypha masses depending on the culture circumstances.

If needed, the thus-formed Basidiomycetes-X hypha masses are dried, and the resultant dry product is pulverized, to thereby yield a dry powder of Basidiomycetes-X (hereinafter referred to as a Basidiomycetes-X dry powder). The pyrrole compound of the present invention can be extracted from the Basidiomycetes-X dry powder and isolated and purified.

No particular limitation is imposed on the method of extracting the pyrrole compound of the present invention from Basidiomycetes-X dry powder. In one mode of efficiently extracting cell contents from Basidiomycetes-X hypha masses, preferably, cell walls of the Basidiomycetes-X hypha masses are optionally broken by means of freezing, for example. The product is thawed and broken by means of a mixer or the like, and an extraction liquid (i.e., extract) is yielded.

No particular limitation is also imposed on the solvent for use in extraction, and water, a lower alcohol, etc. may be used. Also, an extraction solvent further containing an acid, an alkali, or another additive may be used. Extraction is performed at ambient temperature or under heating or pressure. In one general mode of extraction, Basidiomycetes-X hypha masses are boiled in water for extraction. In an alternative mode, a broken product of Basidiomycetes-X hypha masses is mixed with water or an aqueous mixture containing an alcohol or an alkali, and the resultant mixture is pressurized at, for example, about 100 MPa to about 700 MPa, preferably about 300 MPa to about 600 MPa, for extraction.

An example of the extraction method will next be described. Firstly, frozen Basidiomycetes-X hypha masses are thawed at ambient temperature and broken by means of a mixer. The ratio in amount of the broken Basidiomycetes-X hypha mass to that of water (extraction solvent) is adjusted to, for example, about 1:5. Specifically, the broken Basidiomycetes-X hypha masses (50 g) are placed in a glass bottle, and water (250 mL) is added to the bottle. The bottle is closed. Separately, a towel is placed on the bottom of a pan, and water is poured onto the towel. The glass bottle accommodating the broken product of the Basidiomycetes-X hypha masses is mounted on the towel, and the pan is heated to boil water. Heating is continued for 90 minutes after boiling, and the contents of the glass bottle are cooled. Through phase separation, an extract (Basidiomycetes-X extract composition) and a residue (Basidiomycetes-X extraction residue) are yielded. The pH of the extract is, for example, 6.3 to 6.5. Instead of a broken product of Basidiomycetes-X hypha masses, a Basidiomycetes-X dry powder may be used. In this case, the Basidiomycetes-X dry powder is statically cultured in an aqueous medium for 4 hours to 6 hours, while the medium is suitably stirred. The product is subjected to phase separation, to thereby yield an extract and an extraction residue.

The thus-obtained extract is optionally concentrated, to thereby provide a concentrated extract, which may also be used. No particular limitation is imposed on the extract concentration method, and one exemplary mode is as follows.

Firstly, the obtained extract is transferred to a beaker and is concentrated through heating and evaporation. In the course of concentration, the color of the extract changes from light beige to brown, and vigorous effervescence is observed. Evaporation/concentration is further performed. When the extract assumes a tar-like liquid having a pH of 4.9 and a density of 1.25 g/cm³, concentration is stopped. The thus-concentrated extract gives off a soy source-like flavor. At this timing, the average yield of the concentrated extract from the Basidiomycetes-X hypha masses is 12%. Since the viscosity of the thus-obtained concentrated extract steeply increases during cooling of the extract, the extract must be transferred to a storage container immediately after termination of concentration. After cooling, the concentrated extract placed in the storage container is preferably stored in a frozen state.

From the thus-obtained extract or concentrated extract (i.e., a Basidiomycetes-X extract composition), the pyrrole compound of the present invention can be isolated and purified. No particular limitation is imposed on the isolation/purification method, and a customary technique may be employed.

As disclosed in Patent Document 1, the Basidiomycetes-X dry powder and the extract composition thereof (i.e., Basidiomycetes-X extract composition) exhibit high anti-oxidative power and OH radical-scavenging activity, whereby the powder and composition are expected to exhibit an anti-aging action and the like. Also, since the powder and composition have an immunomodulating action, they are suitably used as an immunoactivator or the like. Furthermore, as disclosed in Patent Document 2, the Basidiomycetes-X dry powder and the extract composition thereof exhibit excellent effects of ameliorating/preventing an atopic disease. Thus, they are useful as a composition for atopic diseases.

In addition, “General meeting of the Society of Echigoshirayukidake Researches (2016), 7th Seminar and Sessions” reports “the effect of Echigoshirayukidake on non-alcoholic steatohepatitis (NASH) by Basidiomycetes-X dry powder or Basidiomycetes-X extract composition.” According to the report, Basidiomycetes-X dry powder or Basidiomycetes-X extract composition can ameliorate NASH and prevent aggravation of NASH to cirrhosis or hepatocellular carcinoma.

In the cases where the Basidiomycetes-X dry powder or the Basidiomycetes-X extract composition is used for the aforementioned anti-aging, amelioration of NASH, prevention of aggravation of NASH to cirrhosis or hepatocellular carcinoma, etc., or used as the aforementioned immunoactivator, composition for atopic diseases, etc., the Basidiomycetes-X dry powder or extract composition may be in the form of dry powder or extract liquid. If required, the Basidiomycetes-X extract composition may be dried and formed into a shape of powder, granule, tablet, capsule, solution, gel, etc. Alternatively, the Basidiomycetes-X dry powder may be used as is. Notably, no particular limitation is imposed on the Basidiomycetes-X dry powder content or the Basidiomycetes-X extract composition content of each product, and the content may be appropriately tuned in accordance with the purpose of use.

The Basidiomycetes-X dry powder or the Basidiomycetes-X extract composition may be processed into various food compositions and beverage compositions of any of the aforementioned forms. If such compositions are subjected to optional treatment, they may provide supplements, beverages, etc. Notably, no particular limitation is imposed on the amount of the Basidiomycetes-X dry powder or the Basidiomycetes-X extract composition in each of the food compositions and beverage compositions, and the amount may be suitably tuned in accordance with need.

In the cases where the Basidiomycetes-X dry powder or the Basidiomycetes-X extract composition is used for the prevention and treatment of atopic diseases, NASH, and the like, or for the prevention of cirrhosis or hepatocellular carcinoma, and the like, no particular limitation is imposed on the method of causing a patient to take the powder or composition, and the effective amount thereof may be appropriately determined depending on the extent of the target diseases, the symptoms attributed to the diseases, and other factors. The patient may take the powder or composition in the thus-determined amount. From the viewpoint of easiness in taking daily life, oral ingestion is preferred. In one mode of oral ingestion in the above therapeutic and prophylactic methods, a patient is caused to take a Basidiomycetes-X extract composition dry powder preferably formed into tablets each having a dose of 200 mg to 300 mg, one to thrice per day, preferably thrice per day. No particular limitation is imposed on the ingestion period, but the period is preferably long; for example, preferably 8 weeks or longer, more preferably 16 weeks or longer. Alternatively, the Basidiomycetes-X dry powder may be in the form of tablet or liquid such as syrup, for ingestion.

As mentioned above, the pyrrole compound of the present invention can be isolated and purified from the Basidiomycetes-X extract composition. Alternatively, the compound may be synthesized through a specific method. For example, the compound may be synthesized through a method of synthesizing 4-[formyl-5-(hydroxymethyl)-1H-pyrrol-1-yl]butanoic acid (i.e., the pyrrole compound represented by the following formula (2)), which is a known compound and which is an analogue to the pyrrole compound of the present invention. Notably, the method of synthesizing the pyrrole compound represented by the following formula (2) is disclosed in, for example, M. Ninomiya et. al, B. B B., 1992, 56, p. 806 to p. 807; “Natural Product Source,” Y.-W. Chin et. al., Med. Chem. Lett., 2003, 13, p. 79 to p. 81; and other documents.

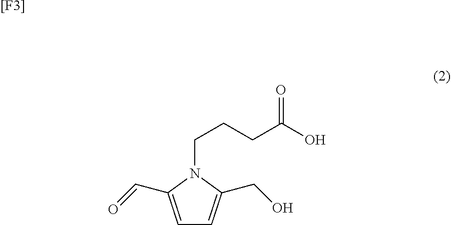

The pyrrole compound represented by the above formula (1) can be synthesized through the following procedure.

Firstly, 4-aminobutanamide (190.8 mg, 1.87 mmol), D-glucose (356.5 mg, 1.98 mmol), triethylamine (250 mg, 2.47 mmol), and oxalic acid (193.5 mg, 2.15 mmol) are dissolved in dimethyl sulfoxide (5 mL). The resultant mixture is caused to react at 90° C. for 30 minutes under stirring. The reaction is cooled to room temperature, and then water (20 mL) is added thereto. The mixture is subjected to extraction with ethyl acetate (20 mL) three times. The ethyl acetate layer is dried over sodium sulfate anhydrate, and subjected to distillation under reduced pressure, to thereby obtain a crude product. The product is purified through preparative thin layer chromatography (PTLC) layer chromatography) with a mixture of hexane-ethyl acetate-methanol (4:4:2 (v/v)) as a developing solvent. As a result, about 7 mg of an amide form is recovered (yield: about 2%). The amide form is the pyrrole compound of the present invention.

### EXAMPLES

The pyrrole compound of the present invention will next be described in more detail by way of examples.

### Production Example 1

<Separation from Hypha Masses>

### (1) Preparation of Culture Medium

A PSA medium and a PDA medium having the compositions shown in Table 1 were prepared. Each medium was dispensed into a test tube or an Erlenmeyer flask, which was stoppered with Silicosen (or a cotton plug). These media were sterilized with high-pressure vapor at 121° C. for 20 minutes in an autoclave. In the case of a test tube, a hot medium after sterilization was formed into a slant medium, whereas in the case of an Erlenmeyer flask, a sterilized medium was allowed to stand to form a plate medium.

TABLE 1

| PSA medium | PDA medium |
|---|---|
| Petro 200 g (20 min-boil/extract) | Petro 200 g (20 min-boil/extract) |
| Sucrose 20 g | Glucose 20 g |
| Agar 15 g | Agar 15 g |
| Total volume 1 L | Total volume 1 L |

(2) Separation from Hypha Masses

Larger Basidiomycetes-X hypha masses were broken manually, and slices were cut from Basidiomycetes-X sections with a scalpel which had been flame-sterilized and cooled. The PSA medium and the PDA slant medium of (1) were each inoculated with the Basidiomycetes-X slices by means of tweezers which had been flame-sterilized and cooled. This procedure was performed under aseptic conditions in an aseptic box or a clean bench.

### (3) Culturing in Agar Medium for Production of Hypha Masses

Potato dices (1 cm×1 cm) (200 g) were boiled in purified water for 20 minutes and then cooled. The broth was separated from the solid. To a mixture of the broth (potato extract), sucrose (20 g), and agar (1 g, 0.1%), distilled water was added, so that the total volume was adjusted to 1 L, to thereby prepare an agar medium. Although a conventional agar medium has an agar concentration of 1.5 to 2.0 (i.e., 15 g to 20 g based on 1 L of the medium), the agar concentration of this medium was adjusted 0.1%, for facilitating isolation of cultured hypha masses from the agar medium and maintaining the physical strength of Basidiomycetes-X slices, which readily cause sedimentation in a liquid culture medium. The 0.1% agar medium (each 5 mL) was dispensed into test tubes, which were stoppered with Silicosen. These media were sterilized with high-pressure vapor at 121° C. for 20 minutes in an autoclave. Thereafter, a slice was cut from the Basidiomycetes-X hypha masses in culturing on the slant medium of Production Example 1. This operation was performed in an aseptic box after aseptic treatment. The slice was inoculated to the 0.1% agar medium. The inoculum was cultured in an incubator at 24° C., and was found to generate the organism in 24 to 48 hours. After generation of the organism, culture was continued at 24° C. As a result, hyphae grew on the agar media in 14 days.

### Production Example 2

### <Culturing in Sawdust Medium for Production of Hypha Mass>

### (1) Culturing of Seed Fungus

Water was added to sawdust (1 L), defatted bran (15 g), wheat bran (15 g), and SANPEARL (hypha activator, product of Nippon Paper Industries) (5 g), and the mixture was vigorously stirred. This mixture for culture was adjusted such that when it was firmly gripped, water exuded (water content of the mixture: about 70%), whereby a sawdust medium was prepared. This culture medium was placed in an Erlenmeyer flask, which was stoppered with Silicosen. Then, the Erlenmeyer flask was subjected to high pressure steam sterilization in an autoclave for 40 minutes at 121° C. Twenty-four hours after the sterilization, Basidiomycetes-X hyphae during culture on the slant media in Production Example 1 were inoculated into the sawdust medium within an aseptic box through an aseptic operation. The inoculation was carried out such that no damage was caused to the hyphae, with a sterilized triangular knife being used to cut off a part of the slant medium. The density of the inoculation was adjusted to 20% to 30% of the surface area of the sawdust medium. When the inoculum was cultured at 24° C., the organism was generated in 3 days (in 5 days at the latest). After a lapse of 30 days, the sawdust medium in the Erlenmeyer flask was full of hyphae.

### (2) Generation of Hypha Mass

A sawdust medium was prepared in the same manner as employed in (1). This culture medium was placed in a polypropylene bottle, which was stoppered, and subjected to high pressure steam sterilization in an autoclave for 40 minutes at 121° C. Twenty-four hours after the sterilization, the seed organism cultured in (1) was inoculated into the sawdust medium placed in the polypropylene bottle through an aseptic operation within an aseptic box after aseptic treatment. The density of the inoculation was adjusted such that the surface area of the sawdust medium was virtually covered with the inoculum. When the inoculum was cultured at 24° C., the organism was generated in 48 hours. After a lapse of 60 days, the entire sawdust medium within the polypropylene bottle was full of hyphae. After a further lapse of 40 to 50 days, hyphae spread on the inner wall of the polypropylene bottle, forming mycelial strands. When culture was continued further, hypha masses were formed.

### Production Example 3

### <Production of Basidiomycetes-X Dry Powder>

In order to cause damage to the cell walls of the hyphae and facilitate the leaching-out of the cell contents, fresh Basidiomycetes-X hypha masses obtained in Production Example 2 were frozen. The thus-frozen Basidiomycetes-X hypha masses were thawed at ambient temperature, and crushed by means of a mixer. The product is dried to form a powder (hereinafter referred to as “Basidiomycetes-X dry powder”).

### Example 1

The Basidiomycetes-X dry powder (50 g) produced in Production Example 3 was subjected to extraction with distilled water (500 mL), to thereby recover 430 mL of aqueous extract (16 g of a freeze-dry product was obtained through freeze-drying the aqueous extract). Subsequently, ethanol (160 mL) was added to the aqueous extract (430 mL), whereby the mixture was separated into a supernatant (540 mL) and precipitates (1.72 g) (12.4 g of solid was obtained through freeze-drying the supernatant (540 mL)). Then, the supernatant (540 mL) was subjected twice to extraction with ethyl acetate (360 mL), to thereby separate the water-containing organic layer. The thus-isolated organic layer (920 mL) of the water-containing organic layer was concentrated and dried, to thereby yield 0.84 g of a dry solid. Separately, when the aqueous layer (270 mL) was freeze-dried, 10.6 g of a solid was produced. From the amount of recovered extraction fractions (precipitate, organic layer, and aqueous layer), ratio of recovery of each extraction fraction with respect to the water extract as 100% was calculated. Table 2 shows the results.

TABLE 2

| | Amount of recovery | Ratio of Recovery |
|---|---|---|
| | (g) | (%) |
| Precipitate | 1.72 | 10.75 |
| Org. layer | 0.84 | 5.25 |
| Aq. layer | 10.6 | 66.25 |
| Total | 13.16 | 82.25 |

### Example 2

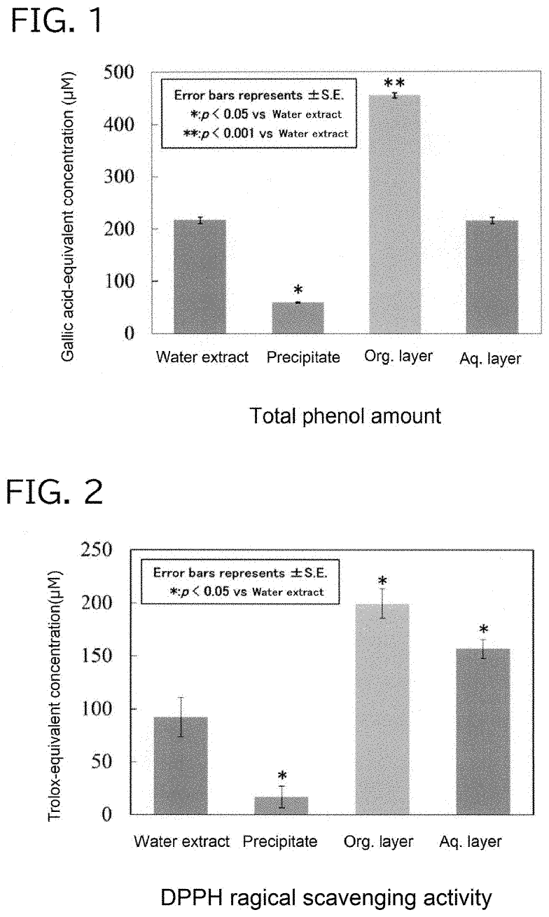

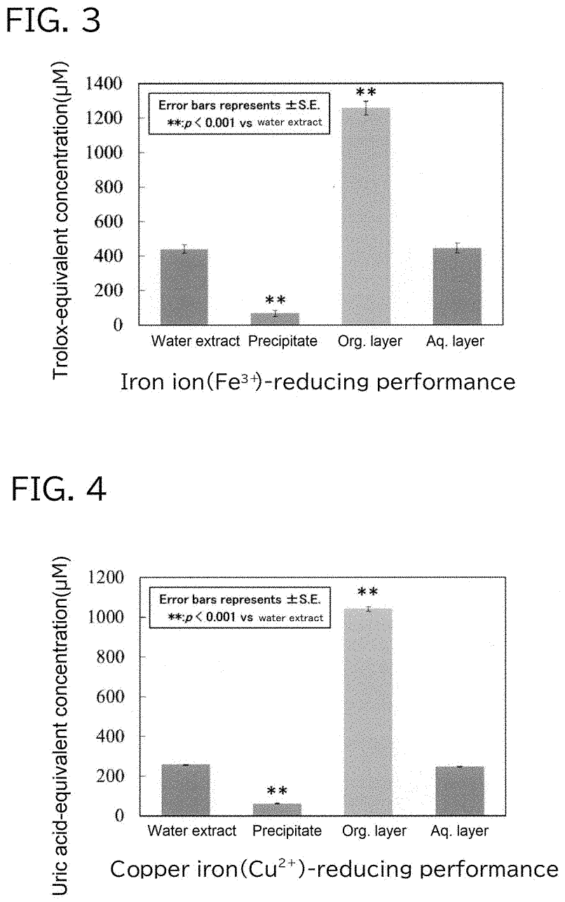

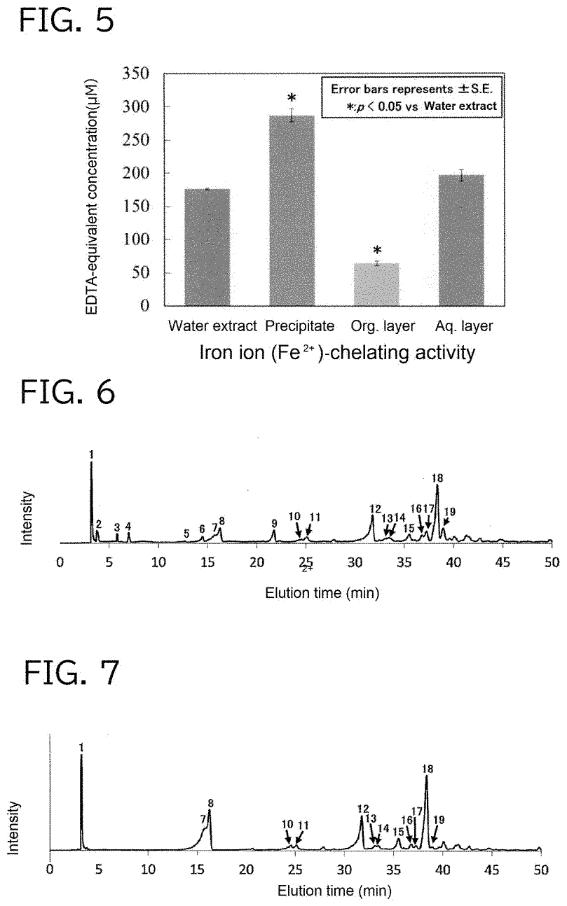

Each of the extraction fractions obtained in Example 1 was analyzed through five assay systems, to thereby evaluate antioxidation ability. FIGS. 1 to 5 show the results. FIG. 1 is a graph showing the total phenol amount in each extraction fraction. The total phenol amount was determined by use of a solution of the extraction fraction having a concentration of 1.25 mg/mL, with reference to a calibration curve drawn by use of gallic acid (i.e., 3,4,5-trihydroxybenzoic acid) as a standard substance. FIG. 2 is a graph showing the DPPH radical scavenging activity of each extraction fraction. The DPPH (2,2-diphenyl-1-picrylhydrazyl) radical scavenging activity was determined by use of a solution of the extraction fraction having a concentration of 2.5 mg/mL, with reference to a calibration curve drawn by use of Trolox (6-hydroxy-2,5,7,8-tetramethylchroman-2-carboxylic acid) as a standard substance. FIG. 3 is a graph showing the iron ion-reducing performance of each extraction fraction. The iron ion (Fe³⁺)-reducing performance was determined by use of a solution of the extraction fraction having a concentration of 2.0 mg/mL, with reference to a calibration curve drawn by use of Trolox as a standard substance. FIG. 4 is a graph showing the copper ion (Cu²⁺)-reducing performance of each extraction fraction. The copper ion-reducing performance was determined by use of a solution of the extraction fraction having a concentration of 2.5 mg/mL, with reference to a calibration curve drawn by use of uric acid as a standard substance. FIG. 5 is a graph showing the iron ion-chelating activity of each extraction fraction. The iron ion (Fe²⁺)-chelating activity was determined by use of a solution of the extraction fraction having a concentration of 15 mg/mL, with reference to a calibration curve drawn by use of EDTA (ethylenediaminetetraacetic acid) as a standard substance. Notably, error bars shown in FIGS. 1 to 5 each represent a standard error (S.E.). The symbol “*” shown in any of FIGS. 1 to 5 indicates that an extraction fraction other than a water extract has a significant difference at a significance level of 0.05, and the symbol “**” indicates that an extraction fraction other than a water extract has a significant difference at a significance level of 0.001. The relative activity of each fraction with respect to the water extract was calculated from the results shown in FIGS. 1 to 5. Table 3 shows the results.

TABLE 3

| | Water | | Org. | Aq. |
|---|---|---|---|---|
| | extract | Precipitate | layer | layer |
| DPPH radical-scavenging activity | 1.00 | 0.20 | 2.39 | 1.88 |
| Fe³⁺-reducing ability | 1.00 | 0.15 | 2.85 | 1.01 |
| Cu²⁺-reducing ability | 1.00 | 0.24 | 4.06 | 0.97 |
| Fe²⁺-chelating activity | 1.00 | 1.63 | 0.36 | 1.12 |

As shown in Table 3, the organic layer exhibited high DPPH radical scavenging activity, Fe³⁺-reducing ability, and Cu²⁺-reducing ability. Thus, high antioxidation ability was confirmed. However, the organic layer exhibited an Fe²⁺-chelating activity which was lower than that of the precipitate or the aqueous layer. Thus, low antioxidation ability was confirmed.

### Test Example 1

Based on the evaluation results of Example 2, the organic layer was analyzed through high performance liquid chromatography (HPLC), so as to determine the number and relative amounts of compounds present in the organic layer exhibiting high antioxidation ability. The analysis was performed at a detection wavelength of 260 nm and 290 nm, for an elution time of 0 to 50 minutes. The results of the analysis of Test Example 1 are shown in FIGS. 6 and 7. FIG. 6 is an HPLC chromatogram of an organic layer (detection wavelength: 260 nm), and FIG. 7 is an HPLC chromatogram of an organic layer (detection wavelength: 290 nm). As shown in FIGS. 6 and 7, at least 19 peaks were detected in each chromatogram. Among them, peak 12 and peak 18 exhibited relatively high intensity.

### Test Example 2

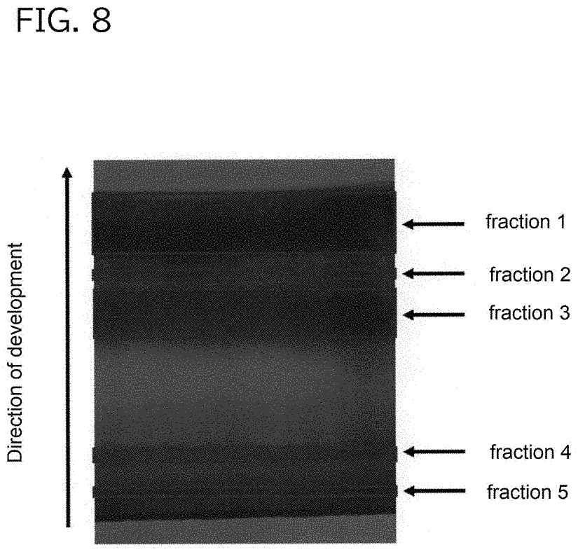

Based on the analytical results of Test Example 1, the organic layer was subjected to preparative thin layer chromatography (PTLC), to thereby isolate/purify compounds present in the organic layer. Specifically, a small amount of the organic layer was added dropwise onto a PTLC plate, and the spot was developed under the following conditions, to thereby separate into fractions. FIG. 8 shows the results. FIG. 8 is a PTLC chromatogram of the organic layer. As shown in FIG. 8, the spot was found to be separated into about 5 parts (fractions 1 to 5).

### [Ptlc Conditions]

PTLC plate: PLC Silica gel 60 F₂₅₄, 2 mm, 20×20 cm, product of Merck
Developing solvent: chloroform 120 mL/methanol 80 mL
Detection wavelength: UV 254 nm
Sample amount: 100 mg/mL methanol (1 mL)

Based on the results of FIG. 8, R_{f}, the amount of the recovered organic layer, and ratio of recovery of each fraction were determined. The ratio of recovery was calculated from the amount of the recovered organic layer, with respect to the amount of organic layer as 100%. Table 4 shows the results.

TABLE 4

| | | Amount of recovery | Ratio of recovery |
|---|---|---|---|
| | R_{f} | (mg) | (%) |
| Org. layer | — | 202.3 | 100 |
| fraction 1 | 0.875 | 19.9 | 9.8 |
| fraction 2 | 0.706 | 3.9 | 1.9 |
| fraction 3 | 0.563 | 10.1 | 5.0 |
| fraction 4 | 0.188 | 6.7 | 3.3 |
| fraction 5 | 0.0625 | 16.0 | 7.9 |
| All fractions | — | 56.6 | 28.0 |

As shown in FIG. 8 and Table 4, a number of compounds were found to be present in fractions 1 and 3, as compared with fractions 2, 4, and 5.

### Test Example 3

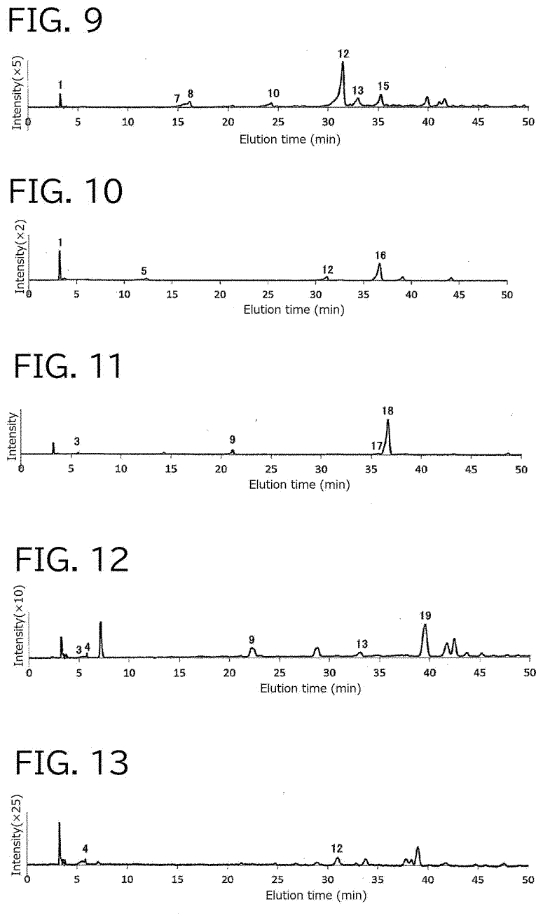

Based on the analytical results of Test Example 2, each fraction was analyzed through HPLC. In Test Example 3, the relative amounts of compounds (proportions) corresponding to each of the peaks shown in Test Example 1 were determined, in consideration of possibility of decomposition of compounds during the course of separation of the organic layer into the fractions. The HPLC analysis was performed at a detection wavelength of 260 nm for an elution time of 0 to 50 minutes. The results of analysis in Test Example 3 are shown in FIGS. 9 to 13. FIGS. 9 to 13 are HPLC chromatograms of fractions 1 to 5. As shown in FIGS. 9 to 13, a compound corresponding to peak 12 (compound 12) was found to be predominantly present in fraction 1, and a compound corresponding to peak 18 (compound 18) was found to be predominantly present in fraction 3.

Peaks shown in HPLC chromatograms of FIGS. 9 to 13 were spectrally analyzed. In each fraction, the elution time, maximum absorption wavelength (λₘₐₓ), and shoulder peak of each compound corresponding to each peak were determined. Table 5 shows the results.

TABLE 5

| | fraction | | | | | |
|---|---|---|---|---|---|---|
| Peak | 1 | 2 | 3 | 4 | 5 | λₘₐₓ (shoulder) |
| No. | Elution time (min) | | | | | (nm) |
| 1 | 3.24 | 3.23 | | | | 288 |
| 2 | | | | | | 260 |
| 3 | | | 5.71 | 5.79 | | 260 |
| 4 | | | | 6.97 | 7.02 | 260 |
| 5 | | 12.35 | | | | 260 |
| 6 | | | | | | 268, 296 |
| 7 | 15.6 | | | | | 296 |
| 8 | 16.19 | | | | | 296 |
| 9 | | | 21.16 | 21.29 | | 260 |
| 10 | | | | | | 296 |
| 11 | | | | | | 288 |
| 12 | 31.45 | | | | 31.52 | 296(260) |
| 13 | 32.94 | 31.14 | | 32.69 | | 296(266) |
| 14 | | | | | | 296(264) |
| 15 | 35.26 | | | | | 296 |
| 16 | | 36.66 | | | | 263, 296 |
| 17 | | | 35.69 | | | 264, 296 |
| 18 | | | 36.69 | | | 296(260) |
| 19 | | | | 35.58 | | 248, 304 |

As shown in Table 5, similarity in structure between compounds each corresponding to each peak was found through spectral analysis. Particularly, spectral properties of compounds 12 and 18 (maximum absorption wavelength and shoulder) indicate similarity in structure.

### Referential Example 1

Compound 18, which was present in fraction 3 at high content as observed in Test Example 3, was subjected to structure analysis. Firstly, the organic layer was separated into fractions through PTLC under the same conditions as employed in Test Example 2. Among the recovered fractions, fraction 3 was further purified through HPLC, to thereby yield compound 18. The molecular weight of compound 18 was determined through mass spectrometry (MS), and the structure of compound 18 was characterized through nuclear magnetic resonance (NMR).

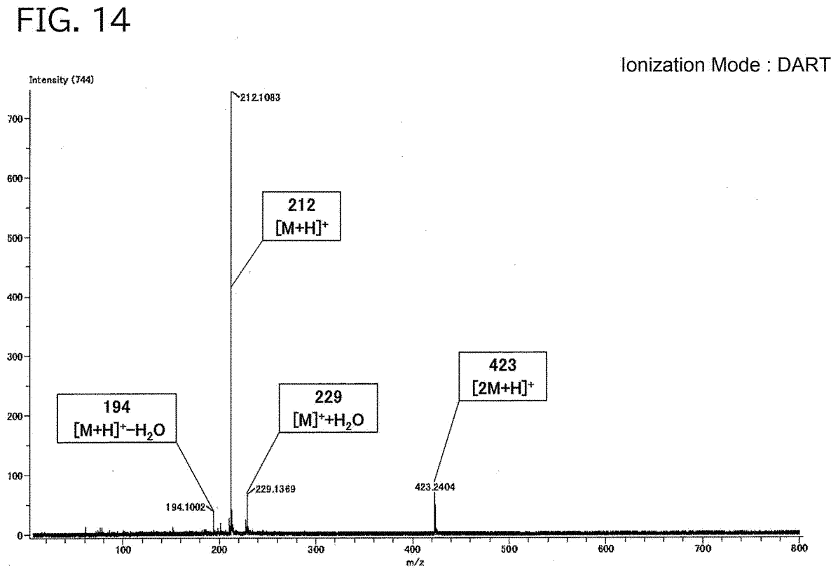

The molecular weight of compound 18 was determined through FAB-HRMS analysis and DART-MS analysis. In the FAB-HRMS analysis, a high resolution mass spectrometer (HRMS) was employed, and a fast atom bombardment (FAB) technique was employed for ionization. In the DART-MS analysis, a DART (direct analysis in real time) ion source was used for ionization in the mass spectrometer. Table 6 shows the results of the FAB-HRMS analysis, and FIG. 14 shows the results of DART-MS analysis. FIG. 14 is a DART-MS spectrum of fraction 3. As shown in Table 6 and FIG. 14, the molecular weight of compound 18 was M+1(H), with excess of 1 mass. However, since the error ratio was very small, the molecular formula was determined as “C₁₀H₁₄O₄N.” Notably, in the milli mass unit (mmu) of Table 6, the relation: 1 u=1 Da is applied. The unit Dalton (Da) represents unified atomic mass unit and a non-SI unit that is accepted for use with SI.

TABLE 6

| | Err | | |
|---|---|---|---|
| Observed m/z | (ppm/mmu) | U.S. | Composition |
| 212.0913 | −4.6/−1.0 | 4.5 | C₁₀H₁₄O₄N |

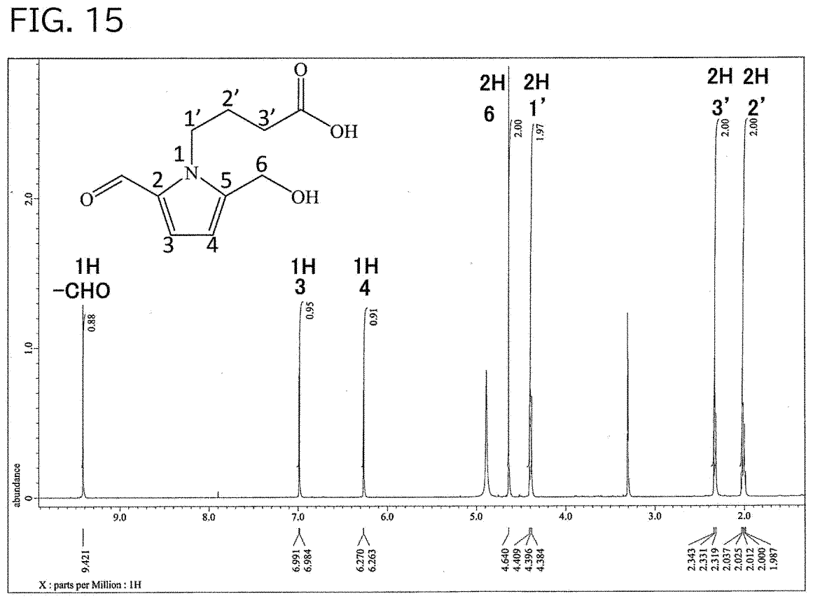

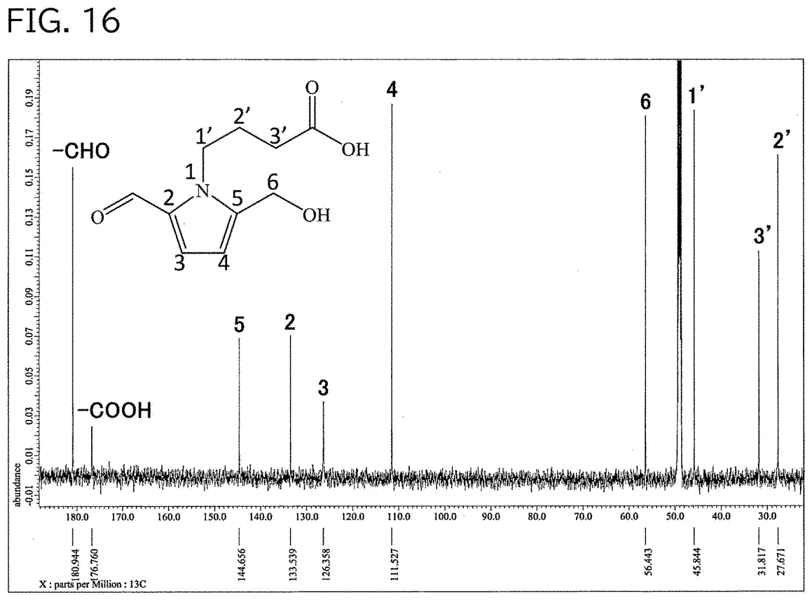

The structure of compound 18 was determined by means of a nuclear magnetic resonance spectrometer with heavy methanol (CD₃OD) as a deuterated solvent for NMR measurement. FIGS. 15 and 16 show the results of structure analysis through NMR. FIG. 15 is a ¹H-NMR spectrum of fraction 3, and FIG. 16 is a ¹³C-NMR spectrum of fraction 3. As shown in FIGS. 15 and 16, compound 18 was found to be represented by the following structural formula (2). That is, compound 18 was identified as 4-[formyl-5-(hydroxymethyl)-1H-pyrrol-1-yl]butanoic acid.

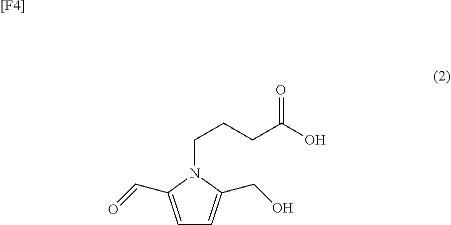

### Example 3

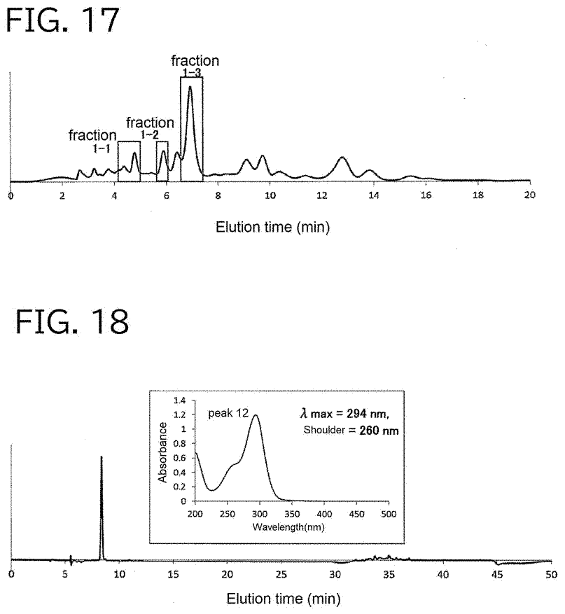

Compound 12, which was present in fraction 1 at high content as observed in Test Example 3, was subjected to structure analysis. Firstly, the organic layer was separated into fractions through PTLC under the same conditions as employed in Test Example 2. Among the recovered fractions, fraction 1 was further purified through reverse phase HPLC, to thereby recover new fractions. FIG. 17 shows the results. The reverse phase HPLC was performed under the following conditions. FIG. 17 is a preparative HPLC chromatogram of fraction 1. As shown in FIG. 17, separated three fractions (Fr1-1, Fr1-2, and Fr1-3) exhibited predominant peaks, and the peak attributed to fraction 1-3 (Fr1-3) was the highest.

### [Reverse Phase HPLC Conditions]

Column: InertSustain, pore size 5 μm, diameter 14 mmX150 mm, product of GL Science
Sample: fraction 1 in 30% aqueous methanol Injection: 200 μL
Flow rate: 5.5 mL/minute
Detection wavelength: 260 nm
Eluent: 30% aqueous methanol (0.1% acetic acid)

### Example 4

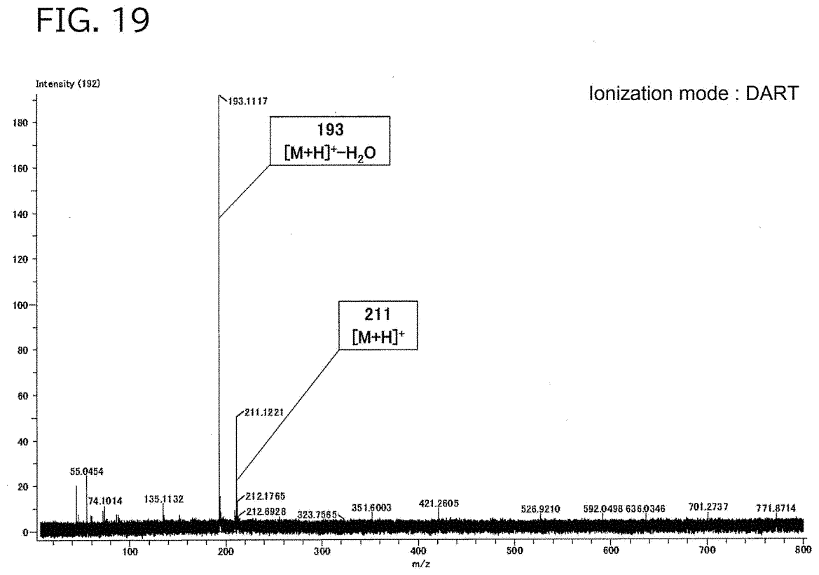

Based on the results of Example 3, fraction 1-3 was analyzed through HPLC. FIG. 18 shows the results. The HPLC was performed under the following conditions. FIG. 18 is a HPLC chromatogram of fraction 1-3, with the maximum absorption wavelength of peak 12. As shown in FIG. 18, the sharp peak of fraction 1-3 was found to correspond to peak 12 detected in Test Example 1. Also, absorption peak 12 was attributed to a compound having a maximum absorption at a wavelength of 297 nm.

### [HPLC Conditions]

Column: Cosmosl 15C18-MS-II, pore size 5 μm, diameter 4.6 mm×150 mm, product of Nacarai Tesque, Inc.
Injection: 20 μL
Flow rate: 0.60 mL/minute
Detection wavelength: 260 nm
Eluent A: 0.1% aqueous acetic acid
Eluent B: 0.1% acetic acid-methanol
Gradient conditions:

0 to 25 min: Eluent A 70%, Eluent B 30%;

25 to 30 min: Linear gradient, Eluent B 30% to 100% (Eluent A 70% to 0%);

30 to 40 min: Eluent B 100%;

40 to 45 min: Linear gradient, Eluent B 100% to 0% (Eluent A 0% to 100%);

45 to 50 min: Eluent A 100%;

55 to 57 min: Eluent A 100% to 70% (Eluent B 0% to 30%); and

57 to 67 min: Eluent A 70%, Eluent B 30%.

### Example 5

The compound corresponding to peak 12 of Example 4 was subjected to DART-MS analysis in the same manner as employed in Referential Example 1, whereby the molecular weight of the compound was determined. FIG. 19 shows the results. FIG. 19 is a DART-MS spectrum of fraction 1-3. As shown in FIG. 19, the compound identified by the DART-MS spectrum of fraction 1-3 was found to be a compound having a molecular weight smaller by 1 mass than that of compound 18 corresponding to the DART-MS spectrum (see FIG. 14).

### Example 6

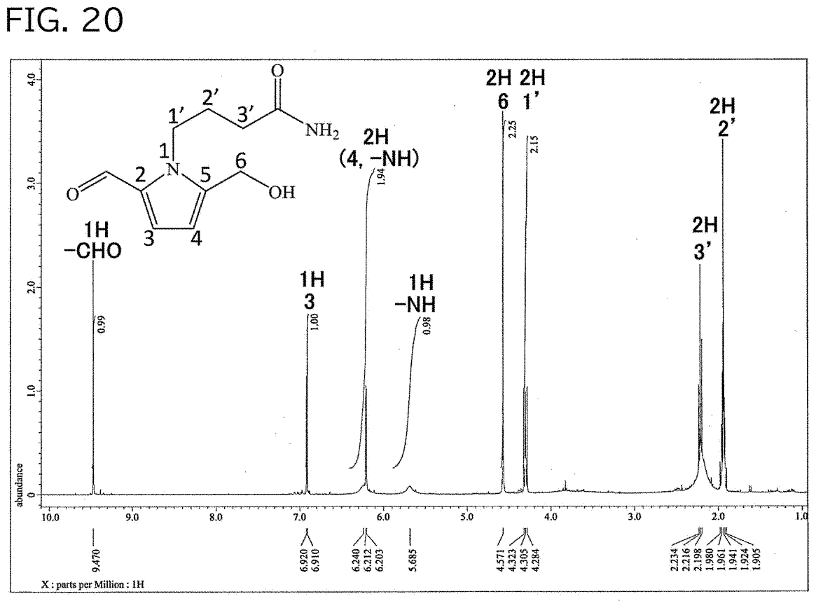

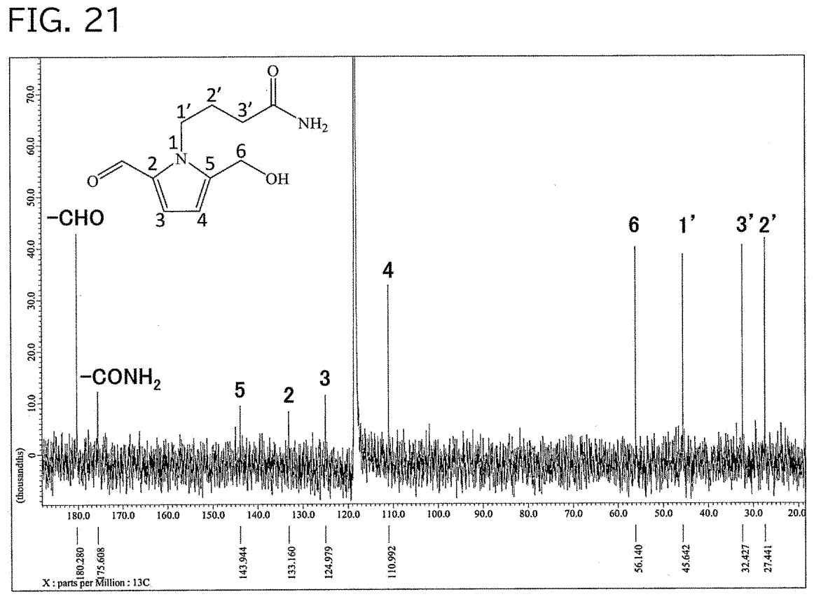

The compound corresponding to peak 12 of Example 4 was analyzed through nuclear magnetic resonance spectroscopy in the same manner as employed in Referential Example 1, and an NMR spectrum of fraction 1-3 was measured. FIGS. 20 and 21 show the results. Notably, heavy acetonitrile (CD₃CN) was used as a deuterated solvent for NMR measurement. FIG. 20 is a ¹H-NMR spectrum of fraction 1-3, and FIG. 21 is a ¹³C-NMR spectrum of fraction 1-3. As shown in FIG. 21, compound 12 was found to have 10 carbon atoms. Also, peaks of the NMR spectrum were assigned with reference to the ¹H-NMR spectrum of FIG. 20.

### Example 7

Based on the results of Example 6, the compound corresponding to peak 12 of Example 4 was subjected to FAB-HRMS analysis in the same manner as employed in Referential Example 1. Table 7 shows the results. As shown in Table 7, in combination with the results of FIGS. 19 to 21, the compound corresponding to peak 12 of Example 4 was found to be represented by the molecular formula “C₁₀H₁₅N₂O₃.”

TABLE 7

| | Err | | |
|---|---|---|---|
| Observed m/z | (ppm/mmu) | U.S. | Composition |
| 211.1081 | −0.8/−0.2 | 4.5 | C₁₀H₁₅N₂O₃ |

Next, the ¹H-NMR spectrum of fraction 3 of FIG. 15 and the ¹³C-NMR spectrum of fraction 3 of FIG. 16 were compared with the ¹H-NMR spectrum of fraction 1-3 of FIG. 20 and the ¹³C-NMR spectrum of fraction 1-3 of FIG. 21. The data of the spectra are shown in Tables 8 and 9 below. As shown in Tables 8 and 9, the ¹H-NMR spectrum and the ¹³C-NMR spectrum of compound 12 have high similarity with those of compound 18, indicating that the chemical structures of two compounds are highly similar to each other.

TABLE 8

| | | δH | HMBC |
|---|---|---|---|
| Position | δC | (J in Hz) | (H to C) |
| 2 | 133.5 | — | — |
| 3 | 126.4 | 6.99, d (4.12) | 2, 4, 5, —CHO |
| 4 | 111.5 | 6.27, d (4.12) | 2, 3, 5 |
| 5 | 144.7 | — | — |
| 6 | 56.4 | 4.64, s | 4, 5 |
| 1′ | 45.8 | 4.40, t* (7.56) | 2′, 3′, 2, 5 |
| 2′ | 27.7 | 2.01, m | 1′, 3′, —COOH |
| 3′ | 31.8 | 2.33, t (7.39) | 1′, 2′, —COOH |
| —CHO | 180.9 | 9.42, s | 2 |
| —COOH | 176.8 | — | — |

*triplet-like coupling

TABLE 9

| | | δH | HMBC |
|---|---|---|---|
| Position | δC | (J in Hz) | (H to C) |
| 2 | 133.2 | — | — |
| 3 | 125.0 | 6.92, d (4.12) | 2, 4, 5 |
| 4 | 111.0 | 6.21, d (4.12) | 2, 3, 5 |
| 5 | 143.9 | — | — |
| 6 | 56.1 | 4.57, s | 4, 5 |
| 1′ | 45.6 | 4.31, t* (7.33) | 2′, 3′, 2, 5 |
| 2′ | 27.4 | 1.94, m** | 1′, 3′, —CONH₂ |
| 3′ | 32.4 | 2.22, t (7.20) | 1′, 2′, —CONH₂ |
| —CHO | 180.3 | 9.47, s | 2 |
| —CONH₂ | 175.6 | — | — |
| —NH₂ | — | 6.24, brd s | — |
| | — | 5.69, brd s | — |

*triplet-like coupling
**multiplet peaks including solvent (CH₃CN) peak

As described hereinabove, compound 12 was found to be represented by the following structural formula (1). The skeleton of the structure is essentially the same as that of compound 18 (see formula (2)), except that the terminal carboxylic acid residue of compound 18 is converted into a carboxamide residue.

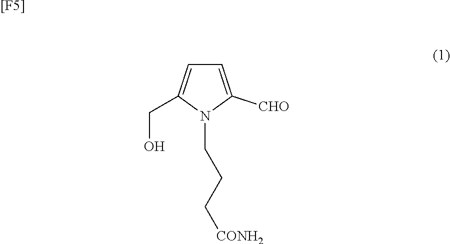

### INDUSTRIAL APPLICABILITY

The Basidiomycetes-X dry powder or the Basidiomycetes-X extract composition containing the pyrrole compound of the present invention is originated from a natural product. Therefore, the Basidiomycetes-X product has high safety and possibly does not provide side effects during a long consumption period. That is, in the cases where the Basidiomycetes-X dry powder or extract composition is used for anti-aging, amelioration of NASH, prevention of aggravation of NASH to cirrhosis or hepatocellular carcinoma, etc., or is used as the aforementioned immunoactivator, composition for atopic diseases, etc., the product may be used as a drug, or incorporated into foods and beverages adaptable to long-term oral ingestion, such as supplements for daily use. The way of administration of the product is safe and simple. As a result, the pyrrole compound of the present invention is highly useful in fields including the aforementioned pharmaceutics and foods.

### [Accession Number]

### Basidiomycetes-X FERM BP-10011

### Reference to Microorganism

### Name of Depositary Institution: NITE Patent Microorganisms Depositary (NPMD), Biological Resource Center, National Institute of Technology and Evaluation

### Address of Depositary Institution: #120, 2-5-8 Kazusakamatari, Kisarazu-shi, Chiba 2920818, Japan

### Date of Deposit to Depositary Institution: Feb. 27, 2003

### Accession Number Given by the Institution: FERM BP-10011

1. A pyrrole compound represented by the following formula (1): [chemical structure]
